(19) United States
(12) Patent Application Publication
(10) Pub. No.: US 2019/0290191 A1
(43) Pub. Date: Sep. 26, 2019

(54) Low impact physical activities for reducing risk of cholesterol and fat metabolism disorders in overweight people

(71) Applicant: Gleap Health Technologies Oy, Hamina (FI)

(72) Inventors: Juhani LEPPÄLUOTO, Oulu (FI); Karl-Heinz HERZIG, Oulu (FI); Riikka AHOLA, Oulu (FI); Timo JÄMSÄ, Oulu (FI); Jari JOKELAINEN, Oulu (FI); Sirkka KEINÄNEN-KIUKAANNIEMI, Oulu (FI)

(73) Assignee: Gleap Health Technologies Oy, Hamina (FI)

(21) Appl. No.: 16/438,506

(22) Filed: Jun. 12, 2019

Related U.S. Application Data
(63) Continuation-in-part of application No. 15/034,185, filed on May 4, 2016.

(30) Foreign Application Priority Data
Nov. 4, 2013 (FI) 20130323

(51) Int. Cl. A61B 5/00; G16H 20/30; A61B 5/11; A63B 69/00

(52) U.S. Cl. CPC A61B 5/486; G16H 20/30; A61B 5/1118; A61B 2562/0219; A61B 5/4866; A63B 69/0028; A61B 5/742; A61B 5/1123

(57) ABSTRACT

Discloses are method and device determining threshold values for the volume and intensity of physical activity and the lowest levels of this activity that reduce risk factors in overweight persons who suffer from disorders of sugar and lipid metabolism. The method comprises of registering a person's daily physical activity, comparing it with the measured/predetermined threshold levels and informs the person, if the volume and/or intensity of the physical activity has been sufficient to prevent the effects of disease risk factors. Based on the disclosed methodology, overweight persons having disorders in sugar and lipid metabolism are able to perform physical activity maintaining health and reducing the risk factors. The invention described herein demonstrates a novel opportunity for overweight people having an abnormal blood sugar to use the low intensity physical activity to restrain risk factors related to cardiovascular diseases, diabetes and obesity.

### CROSS-REFERENCE TO RELATED APPLICATIONS

This application is a continuation-in-part patent application claiming the benefit of pending U.S. patent application Ser. No. 15/034,185 filed on May 4, 2016 (published as US 2016/0283696), which is a 35 U.S.C. § 371 submission of PCT/FI2014/000031 filed on Oct. 31, 2014, and claims priority to Finnish patent application 20130323 filed on Nov. 4, 2013, the contents each of which are incorporated herein in their entirety by reference thereto.

### TECHNICAL FIELD

The invention generally relates to health-improving physical exercises. More particularly but not exclusively, this invention relates to practicing low impact physical activity using corresponding determined thresholds for improving sugar, cholesterol and fat metabolism in overweight people/subjects having a sedentary lifestyle.

### BACKGROUND OF THE INVENTION

The use of motion sensors and transduction units to measure physical activity intensity and volume have been presented in US2008/0312560A1, WO02005117703. A method in which physical activity improving cholesterol metabolism in healthy subjects is presented in US 2010/0137107 A1. The earlier invention purposed for healthy subjects related to physical activity reducing blood cholesterol (US 2010/0137107 A1) is not suitable to subjects who have risks of diabetes and/or cardiovascular diseases, as they are not capable to perform prescribed physical activity previously shown to reduce blood cholesterol in healthy subjects.

Overweight and metabolic diseases increase significantly in industrialized countries in which energy rich diets are widely used and energy expenditure remains low. According to the WHO statistics, over 1,400,000,000 of the world population are overweight or obese. Overweight and low physical activity result in an increase in the prevalence of metabolic diseases mainly diabetes and cardiovascular diseases. It is currently estimated that 500,000,000 of the population suffer from type 2 diabetes and the number will be doubled by the year 2030. In addition, atherosclerosis, calcification of blood vessels, due to sedentary lifestyle exposes to deaths for cardiovascular diseases being the most frequent death causes in industrialized countries. The dead roll is presently 15,000,000 annually and is growing every year. Low levels of physical activity is also a dangerous risk factor disability-adjusted life years. According to the present WHO statistics, it is annually responsible for 1,000,000 death cases and loss of 8,000,000 disability-adjusted life years.

Official guidelines for physical activity promoting health are briefly discusses herein. The present physical activity recommendations of WHO, American Diabetes Association and American Heart Association state that healthy adults should perform at least 150 min moderate intensity physical activity every week such as walking at the speed of 5 km/h. This threshold should imply walking 2 km or 3000 steps within 20 min during all days of the week. These recommendations have been developed by reviewing subjects about their physical activity levels and demonstrated that about 50% of the adult population met the guidelines. Later physical activity levels have been studied objectively by accelerometers and the results showed that only less than 5% of the adult population will meet the above-mentioned official guidelines of physical activity. Therefore, all the previous studies about the health effects of physical activity using personal reviews or questionnaires are questionable.

Physical activity may have a positive effect on risks for diabetes, cardiovascular and metabolic diseases. Calcification of blood vessels, high blood sugar, cholesterol and triglycerides are risk factors for diabetes and cardiovascular diseases. These risk factors can be reduced by increased physical activity and weight reduction programs. Increased exercise will lead to decreases in blood LDL-cholesterol and increases HDL-cholesterol (Tannescu et al. JAMA, 288, p. 1994, 2002, Kraus et al., NEJM 347, p. 1483, 2002) all reducing risks of cardiovascular diseases. In a similar way increased weekly exercise and weight reduction programs prevented developing of the type 2 diabetes in 60% of patients (Tuomilehto et al. 2002, NEJM 304.1343, Knowler et al. 2002, NEJM 346:393). Fat tissues especially those around the internal organs are body's energy sources and are released to blood circulation during exercises. It has been shown that during exercises fat from the internal organs is first mobilized and starts the reduction of body weight (O'Leary et al., J Appl Physiol 100, p. 1958, 2009).

The results of scientific studies demonstrate that increased physical activity is effective in preventing risk factors of diabetes and cardiovascular diseases and is an important vehicle in weight reduction. However, in none of these cited or other previous studies the exact amounts and intensities of physical activity preventing diabetes or cardiovascular diseases have been described. Our research group is the only one that have measured physical activities by objective methods in order to find out beneficial effects of disease risk factors. Our main goal has been to define the minimum amounts and intensities or threshold levels which will reduce the risks for diabetes and cardiovascular diseases. By using our accelerometer we have previously demonstrated that blood cholesterol decreases by 15% in healthy women as the number of daily steps exceeded 1000 at acceleration of 2.1 g corresponding to walking at the speed of 5 km/h (Vainionpää et al., Me Sci Sport Exerc 39, p. 756, 2007).

### SUMMARY OF THE INVENTION

According to a first aspect of the invention, an apparatus comprising: an accelerometer that continuously registers accelerations maximum values induced by a physical activity of a person, said apparatus is carried by the person during day activities, the person being overweight and having an abnormal blood sugar; a processor; and a memory for storing the registered acceleration maximum values and tangibly storing computer program instructions capable of being executed by the processor, the computer program instructions defining code for: determining selected acceleration maximum values out of the stored registered acceleration maximum values which are within at least one preset acceleration range corresponding to a low intensity physical activity comprising slow walking of said overweight person with a speed below 5 km/hour, the at least one preset acceleration range being below 1.1 g and having a lower limit value and a high limit value; calculating a number of daily steps of the person based on the selected acceleration maximum values, where one step corresponds to one selected acceleration maximum value; comparing a total number of steps for a predetermined period of time comprising one or more days or an average number of steps per day with a predefined threshold level; and providing a recommendation for the person, based on the determining and the comparing, to a display of the apparatus or to a wireless device associated with the person and communicating with the apparatus for presenting the recommendation on a screen of the wireless device, about maintaining or modifying the low intensity physical activity of the person including at least a required number of steps per day corresponding to slow walking of the person with the speed below 5 km/hour, in order to reduce a risk of disorders of sugar, blood cholesterol and fat metabolism of the person.

According further to the first aspect of the invention, the lower limit value may be approximately 0.3 g and the high limit value may be a predefined value from 0.5 g to 1.0 g.

According further to the first aspect of the invention, the at least one preset acceleration range may be 0.3-0.7 g corresponding to the low intensity physical activity comprising slow walking of the person with a speed of approximately 2 to 3 km/hour, wherein the predefined threshold level for the total number of steps per day in the acceleration range of 0.3-0.7 g can be 6520.

Still further according to the first aspect of the invention, the at least one preset acceleration range may be 0.3-0.9 g corresponding to the low intensity physical activity comprising slow walking of said overweight person with a speed below 5 km/hour being approximately 2 to 4 km/hour, wherein the predefined threshold level for the total number of steps per day in the acceleration range of 0.3-0.9 g can be 6790.

According further to the first aspect of the invention, the at least one preset acceleration range can be chosen based on matching clinical characteristics of the person to corresponding clinical characteristics of a plurality of clinical groups to select a corresponding clinical group, for each clinical group at least one corresponding predefined threshold level being stored in the memory, so that the stored at least one corresponding predefined threshold level becomes the predefined threshold level.

According still further to the first aspect of the invention, the at least one preset acceleration range and a corresponding predefined threshold level may be determined by a preliminary study of the physical activity of the person using the computer program instructions defining further code for: determining preliminary selected acceleration maximum values of the person out of the stored registered acceleration maximum values which are within a plurality of acceleration ranges being below the acceleration value of 1.1 g comprising slow walking of said overweight person with the speed below 5 km/hour; calculating a preliminary number of daily steps of the person in each of the plurality of the acceleration ranges based on the preliminary selected acceleration maximum values, where one preliminary step corresponds to one preliminary selected acceleration maximum value, thus providing a distribution of the preliminary number of steps in the plurality of acceleration ranges below the acceleration value of 1.1 g; and matching the provided distribution of the preliminary number of steps to a corresponding distribution out of a plurality of corresponding distributions stored in the memory for a plurality of clinical groups for each of which at least one corresponding predefined threshold level being further stored in the memory, and selecting a corresponding clinical group based on the matching, so that for the selected corresponding clinical group, the at least one corresponding predefined threshold becomes the predefined threshold level. In addition, further matching can be provided based on matching clinical characteristics of the person to corresponding clinical characteristics of the corresponding clinical group out of the plurality of clinical groups, so that both, the provided distribution of the preliminary number of steps and the clinical characteristics of the person, can be used for selecting the corresponding clinical group.

According yet further still to the first aspect of the invention, if the number of selected acceleration maximum values has exceeded the predefined threshold level, the low intensity physical activity has prevented at least partly said disorders in sugar, cholesterol and fat metabolism.

According further still to the first aspect of the invention, the apparatus may further comprise a wireless or wired transceiver for one or more of: 1) sending at least the stored registered acceleration maximum values to an outside computing device for a further analysis, and 2) receiving medical test results related to the sugar, blood cholesterol and fat metabolism of the person to perform the further analysis, wherein the further analysis comprises further comparing said total number of steps in the at least one preset acceleration range being below 1.1 g with the medical test results for providing a further recommendation to the person about maintaining or modifying the physical activities of the person.

Yet still further according to the first aspect of the invention, the apparatus may further comprise a wireless or wired transceiver for one or more of: 1) sending at least the stored registered acceleration maximum values to an outside computing device for a further analysis, and 2) receiving medical test results related to the sugar, blood cholesterol and fat metabolism of the person to perform the further analysis, wherein the further analysis can comprise further comparing said total number of steps in the at least one preset acceleration range being below 1.1 g with the medical test results for providing a further recommendation to the person about maintaining or modifying the physical activities of the person.

Still yet further according to the first aspect of the invention, a method performed by an apparatus comprising: continuously registering acceleration maximum values induced by a physical activity of a person using an accelerometer of the apparatus, where the apparatus is carried by the person during day activities, the person being overweight and having an abnormal blood sugar; storing the registered acceleration maximum values in a memory of the apparatus; determining selected acceleration maximum values out of the stored registered acceleration maximum values which are within at least one preset acceleration range corresponding to a low intensity physical activity comprising slow walking of said overweight person with a speed below 5 km/hour, the at least one preset acceleration range being below 1.1 g and having a lower limit value and a high limit value, as executed by a processor of the apparatus using computer program instructions stored in the memory; calculating a number of daily steps of the person based on the selected acceleration maximum values, where one step corresponds to one selected acceleration maximum value, as executed by the processor of the apparatus using the computer program instructions stored in the memory; comparing a total number of steps for a predetermined period of time comprising one or more days or an average number of steps per day with a predefined threshold level, as executed by the processor of the apparatus using the computer program instructions stored in the memory; and providing a recommendation for the person, based on the determining and the comparing, to a display of the apparatus or to a wireless device associated with the person and communicating with the apparatus for presenting the recommendation on a screen of the wireless device, about maintaining or modifying the low intensity physical activity of the person including at least a required number of steps per day corresponding to slow walking of the person with the speed below 5 km/hour, in order to reduce a risk of disorders of sugar, blood cholesterol and fat metabolism of the person, as executed by the processor of the apparatus using the computer program instructions stored in the memory.

According further to the second aspect of the invention, the lower limit value may be approximately 0.3 g and the high limit value can be a predefined value from 0.5 g to 1.0 g.

Further according to the second aspect of the invention, the at least one preset acceleration range may be 0.3-0.7 g corresponding to the low intensity physical activity comprising slow walking of the person with a speed of approximately 2 to 3 km/hour, wherein the predefined threshold level for the total number of steps per day in the acceleration range of 0.3-0.7 g is 6520.

Still further according to the second aspect of the invention, the at least one preset acceleration range may be 0.3-0.9 g corresponding to the low intensity physical activity comprising slow walking of said overweight person with a speed below 5 km/hour being approximately 2 to 4 km/hour, wherein the predefined threshold level for the total number of steps per day in the acceleration range of 0.3-0.9 g can be 6790.

According further to the second aspect of the invention, the at least one preset acceleration range can be chosen based on matching clinical characteristics of the person to corresponding clinical characteristics of a plurality of clinical groups to select a corresponding clinical group, for each clinical group at least one corresponding predefined threshold level being stored in the memory, so that the stored at least one corresponding predefined threshold level becomes the predefined threshold level.

According still further to the second aspect of the invention, the at least one preset acceleration range and a corresponding predefined threshold level can be determined by a preliminary study of the physical activity of the person using the computer program instructions defining further code for: determining preliminary selected acceleration maximum values of the person out of the stored registered acceleration maximum values which are within a plurality of acceleration ranges being below the acceleration value of 1.1 g comprising slow walking of said overweight person with the speed below 5 km/hour; calculating a preliminary number of daily steps of the person in each of the plurality of the acceleration ranges based on the preliminary selected acceleration maximum values, where one preliminary step corresponds to one preliminary selected acceleration maximum value, thus providing a distribution of the preliminary number of steps in the plurality of acceleration ranges below the acceleration value of 1.1 g; and matching the provided distribution of the preliminary number of steps to a corresponding distribution out of a plurality of corresponding distributions stored in the memory for a plurality of clinical groups for each of which at least one corresponding predefined threshold level being further stored in the memory, and selecting a corresponding clinical group based on the matching, so that for the selected corresponding clinical group, the at least one corresponding predefined threshold becomes the predefined threshold level. In addition, further matching can be provided based on matching clinical characteristics of the person to corresponding clinical characteristics of the corresponding clinical group out of the plurality of clinical groups, so that both, the provided distribution of the preliminary number of steps and the clinical characteristics of the person, can be used for selecting the corresponding clinical group.

According yet further still to the second aspect of the invention, if the number of selected acceleration maximum values has exceeded the predefined threshold level, the low intensity physical activity has prevented at least partly said disorders in sugar, cholesterol and fat metabolism.

Yet still further according to the second aspect of the invention, the method may further comprise: sending, by a wireless or wired transceiver of the apparatus, at least the stored registered acceleration maximum values to an outside computing device for a further analysis using medical test results, related to the sugar, blood cholesterol and fat metabolism of the person to generate further recommendations, and for providing the further recommendations to the person about maintaining or modifying the physical activities of the person.

Still yet further according to the second aspect of the invention, the method may further comprise: receiving, by a wireless or wired transceiver of the apparatus, medical test results related to said sugar, blood cholesterol and fat metabolism of the person to perform a further analysis and to generate further recommendations, and for providing the further recommendations to the person about maintaining or modifying the physical activities of said overweight person.

According to a third aspect of the invention, a non-transitory computer readable storage medium tangibly storing computer program instructions capable of being executed by a computer processor, the computer program instructions defining code for: continuously registering acceleration maximum values induced by a physical activity of a person using an accelerometer of the apparatus, where the apparatus is carried by the person during day activities, the person being overweight and having an abnormal blood sugar; storing the registered acceleration maximum values in a memory of the apparatus; determining selected acceleration maximum values out of the stored registered acceleration maximum values which are within at least one preset acceleration range corresponding to a low intensity physical activity comprising slow walking of said overweight person with a speed below 5 km/hour, the at least one preset acceleration range being below 1.1 g and having a lower limit value and a high limit value; calculating a number of daily steps of the person based on the selected acceleration maximum values, where one step corresponds to one selected acceleration maximum value; comparing a total number of steps for a predetermined period of time comprising one or more days or an average number of steps per day with a predefined threshold level; and providing a recommendation for the person, based on the determining and the comparing, to a display of the apparatus or to a wireless device associated with the person and communicating with the apparatus for presenting the recommendation on a screen of the wireless device, about maintaining or modifying the low intensity physical activity of the person including at least a required number of steps per day corresponding to slow walking of the person with the speed below 5 km/hour, in order to reduce a risk of disorders of sugar, blood cholesterol and fat metabolism of the person, as executed by the processor of the apparatus using the computer program instructions stored in the memory.

### BRIEF DESCRIPTION OF THE DRAWINGS

These and other features and aspects of the present disclosure will become better understood when the following detailed description is read, with reference to the accompanying drawings, in which like characters represent like parts throughout the drawings, wherein:

FIG. 1 is a distribution of mean daily steps for five acceleration classes (ranges) 0.3 g<0.5 g, 0.5 g<0.7 g, 0. 7 g<0.9 g, 0.9 g<1.1 g and >1.1 g for intervention and control groups of subjects being overweight and having blood sugar elevated values, according to an embodiment of the invention;

FIGS. 2A-2D are diagrams presenting differences in amounts of cholesterol (FIG. 2A), LDL-cholesterol (FIG. 2B), triglycerides (FIG. 2C) and visceral fat of inner organs (FIG. 2D) according to daily steps (impacts) of all subjects, according to an embodiment of the invention;

FIG. 3 is a flowchart for demonstrating implementation of an exemplary embodiment of the invention;

4A-4B are exemplary flowcharts for further demonstrating implementation of various embodiments of the invention; and

FIG. 5 is a block-diagram of a system/apparatus for implementing embodiments of the invention.

### DETAILED DESCRIPTION

High occurrence of overweight, obesity, diabetes and cardiovascular diseases in the populations of industrialized countries are major health problems. Precise physical activity guidelines are not available in adequately targeting these health problems. Various embodiment of the invention described herein provides determination of physical activity thresholds that can be used for preventing risks for metabolic diseases such as diabetes and cardiovascular disease by reducing blood cholesterol, triglycerides and visceral fat mass in overweight people/subjects/persons having at least abnormal blood sugar.

Before the present invention submitted herein, no information was available about the exact amount and intensity of physical activity reducing risk factors of diabetes and cardiovascular diseases in subjects suffering from metabolic disorders. The present physical activity guidelines (WHO, American Diabetes Association and American Heart Association) are based on questionnaires and reviews from healthy subjects and are too demanding for most general subjects often having overweight and suffering from metabolic disorders. Blood sugar and cholesterol levels have been previously followed by motion sensors in subjects who have abnormal blood glucose but the sensors have been used only for the following of the physical activity but not for the studies of health effects (Yates et al., Diabetes care 32:1404 2009, and Saito et al. Ann Intern Med, 171, p. 1352, 2011). In healthy subjects the amounts and intensities of physical activity reducing blood cholesterol (Vainionpää et al., Med Sci Sports Exerc. 39, p. 756, 2007, and US patent application publication 2008/0312560 A1) have been solved by accelerometers detecting number of steps and intensity (g-values). For the same subjects the amount and intensity of the physical activity preventing osteoporosis was solved (Vainionpää et a., Int J Osteoporosis 17, p. 455, 2006, and US patent application publication US 2010137107 Al). It should be noted that both studies were performed on the same subjects, to whom physical activity thresholds reducing cholesterol and preventing osteoporosis could be defined simultaneously by the accelerometer.

Accelerometers (patents/patent application publications US 2010191155 A1, EP 0700661 A2, US 2009171614 A1, EP 2210557 A1 and US 2001049470 A1) and a heart rate monitor (US 20110213) have been previously used for measuring energy expenditure of physical activity or following body weight and nutrient intake in healthy subjects and heart rate monitor for protein consumption and recovery in athletes. It should be noted that all the above-mentioned methods measuring energy expenditure are useless for establishing threshold levels improving health. For instance, in a previous study we demonstrated that walking 12982 daily steps at the acceleration range 0.3 - 1.0 g had no effect on blood cholesterol, whereas 1062 daily steps at the acceleration range 1.1-2.4 g reduced blood cholesterol by 15% (Vainionpää et al. 2007, Med Sci Sports Exerc 39,756). 12982 daily steps at 0.3 - 1.0 g stand for 10 MET-units (MET stand for metabolic equivalent), but 1062 daily steps at 1.1-2.4. stand for 1 MET-unit. Therefore, a conclusion was reached that energy expenditure in MET-units or calories which have been used in the above-mentioned references, neither relate to determining thresholds nor health benefits of physical activities in sedentary subjects.

The invention described herein manages physical activity thresholds reducing blood cholesterol, triglycerides, visceral fat as well as reducing sugar disorder in overweight subjects with metabolic disorders. These subjects are not able to reach physical activity thresholds presented in the patent application (publication US 20107137107, and therefore they need their own physical activity thresholds.

In our study related directly and leading to this invention (using intervention with additional exercises) we observed that in overweight subjects/persons having abnormal sugar balance clearly lower intensity levels of physical activity than those given in the official physical activity recommendations reduced risks for diabetes and cardiovascular diseases (also published in Herzig et al., Int. J. Obesity 38 pp. 1089-1996, 2014 after a priority filing date of the parent US application, Ser. No. 15/034,185). For example, it was found that walking, e.g., at accelerations in a range of 0.3-0.7 g corresponding to the walking speed of approximately 2-3 km/h may lead to decreases in blood triglycerides, cholesterols, visceral fat as well as to improve insulin sensitivity as the number of daily steps exceeded 6520. This was a very important finding, since most overweight subjects suffering from abnormal blood glucose are not able to walk at the speed of 5 km/h required in the present official recommendations. Therefore, most of these subjects do not follow the present recommendations. Our accelerometer recording amounts and intensities made it possible to determine exact threshold levels of physical activity promoting health to be used in the reduction of risk factors of metabolic diseases.

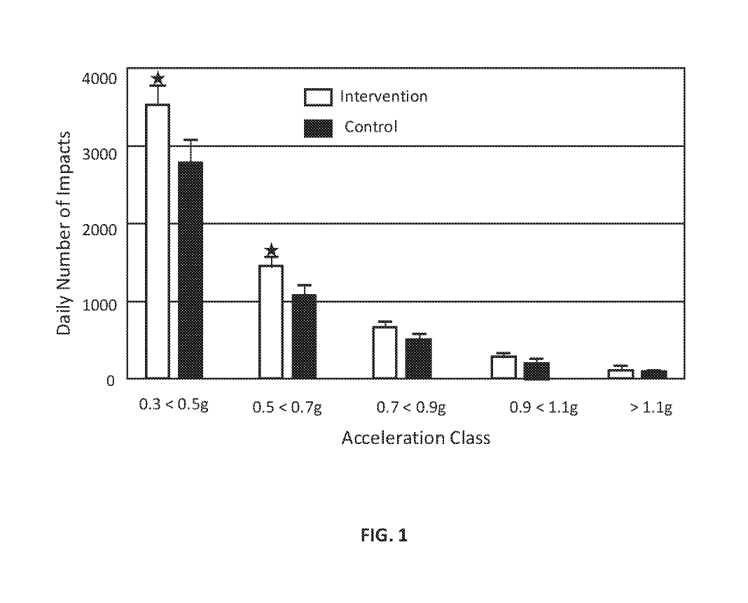

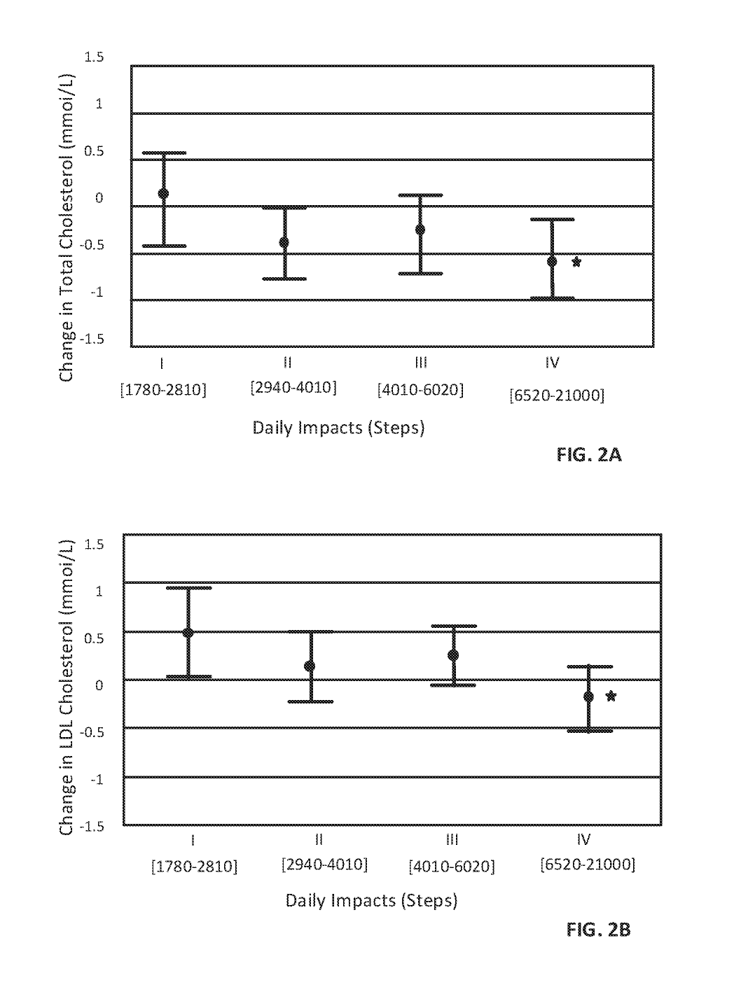

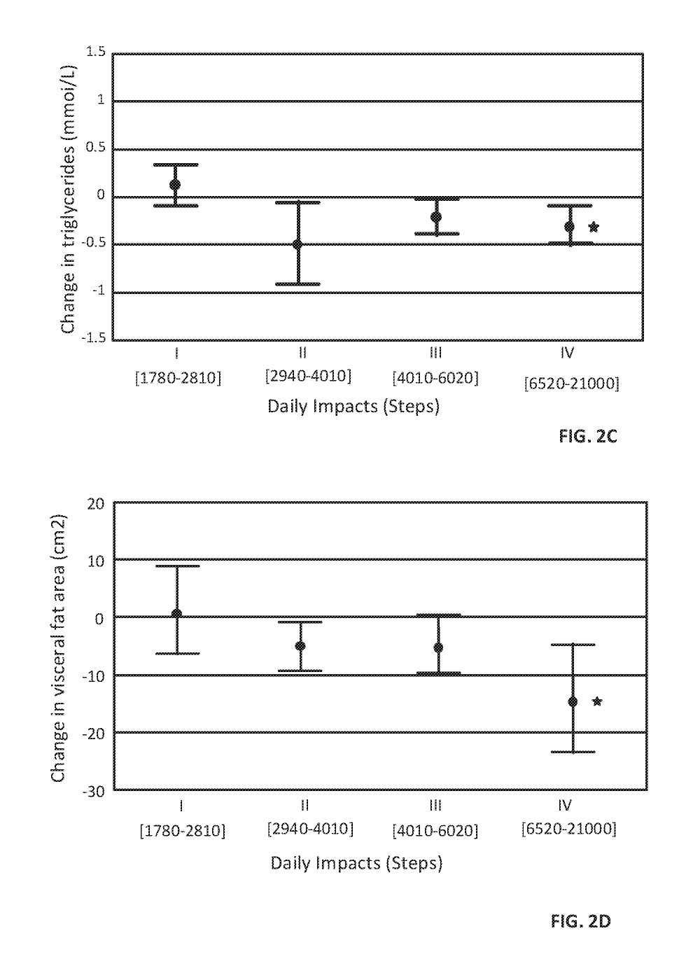

FIGS. 1 and 2A-2D provide a demonstration of the concept according to embodiments of the present invention. The results presented in FIGS. 1 and 2A-2D are generated using data from 68 subjects/persons, each carrying an accelerometer for 3 months. The subjects were overweight men and women aged 30-70 years and had abnormal blood sugar. One half of the subjects participated in a supervised (intervention) exercise on a weekly basis and the other half continued their normal lifestyle. The subjects carried accelerometers during wake-full time on their waist. The acceleration values of their daily numbers were continuously registered. Blood samples were taken at the beginning and at the end of the trial to measure blood sugar, Insulin, cholesterol, LDL-cholesterol and triglycerides. The amount of the visceral fat was measured by a bio-impedance method.

FIG. 1 is a distribution of mean daily steps for five acceleration classes (ranges) of 0.3 g<0.5 g, 0.5 g<0.7 g, 0.7 g<0.9 g, 0.9 g<1.1 g and >1.1 g for intervention and control groups of subjects being overweight and having blood sugar elevated values, according to an embodiment of the invention. The total daily number of steps (average daily step count summarized across all time points; acceleration classes 0.3->1.1) was higher in the intervention than in the control group, median 5576 (range 1196-15 687) vs. 4004 (range 2119-23 088) steps. It is noted that over 80% of the daily steps clustered at the acceleration ranges 0.3-0.7 g and were 5870±3277 (mean±s.d.) in the intervention and 4434±3460 (mean±s.d.) in the control group.

FIGS. 2A-2D are diagrams presenting differences in amounts of cholesterol (FIG. 12A), LDL-cholesterol (FIG. 2B), triglycerides (FIG. 2C) and visceral fat of inner organs (FIG. 2D) based on daily steps (impacts) of all subjects. The steps in the acceleration class/range 0.3-0.7 g were divided by their magnitudes to quartiles (i.e., into four different groups) based on their size, and it is observed that cholesterol, LDL cholesterol, triglycerides and fat content in inner organs degrease when the daily step number is more than 6520 (fourth quartile). This can be a threshold number that needs to be exceeded in order for the exercise to cause positive effects on amounts of cholesterol and triglyceride amounts as well as inner organ's fat amount. In other words, 6520 daily steps is the threshold for physical activity in the acceleration class/range 0.3-0.7 g (corresponding to a walking speed of approximately 2-3 km/hour), the exceeding of which can reduce a total cholesterol by 0.6 mmol/l, LDL-cholesterol by 0.7 mmol/l, triglycerides by 0.4 mmol/l and the area of visceral fat by 12%. Our results show that physical activity volume and intensity measured by the invention described herein explain beneficial health effects observed in cholesterol and triglyceride concentrations and in visceral fat. Corresponding results in subjects with sedentary lifestyle have not been able to obtain by using previous methods. It is noted that a moderate improvement of insulin sensitivity as the number of daily steps exceeded 6520 was also observed.

The data, presented herein in FIGS. 1 and 2A-2D, were analyzed using the SPSS statistical package (PASW statistics 18 for Windows, SPSS Inc., Chicago, Ill., USA). Unpaired two-tailed t-test, Chi-squared-test and w2-test were used to compare participant characteristics at baseline. The efficacy of the intervention on primary and secondary end points was assessed using an unpaired t-test based on the original values or on logarithmic transformations, or a nonparametric test (Mann-Whitney, details in Tables and Figures). Repeated measures analysis of variance was used to determine the effect of the intervention on glucose, insulin and lipid concentrations, body constituents and macronutrients and controlled for potential confounding variables like baseline (model 1), sex, age (model 2, including baseline) and body weight changes (model 3 including baseline, sex and age). Mixed models for repeated measurements were used to analyze weekly impact data. Group randomization was used as a covariant in the analysis of insulin, LDL cholesterol and visceral fat data. We also evaluated the effects of daily total PA (physical activity) on our outcome variables by combining them together from both groups, dividing to quartiles based on the average number of steps per day and comparing the variables between the least and most active quartiles using Dunnett's test.

The data presented in FIGS. 1 and 2A-2D and corresponding analysis is only one example indicating how to practice the invention. For example, in another embodiment, if the chosen acceleration range for monitoring a subject's (person's) low intensity physical activity to be 0.3-0.9 g instead of 0.3-0.7 g, then this will require 1080 more steps, so that extra 270 steps should be added to each of four quartiles. Thus, another threshold in a fourth quartile may be 6790 steps for the monitoring the acceleration range of 0.3-0.9 g which may correspond to a walking speed of approximately 2-4 km/hour. Similar calculation can be made for other acceleration ranges below 1.1 g corresponding to walking speeds of less than 5 km/hour to be chosen for monitoring. For example, the monitored acceleration range can be chosen to be in between approximately 0.3 g (lower limit value) and a high limit value can be chosen to be between 0.5 and 1.0 g. Also, acceleration ranges can be chosen differently than those shown in FIG. 1, so that monitoring acceleration ranges and corresponding thresholds for a number of steps can be chosen to be different using the procedure described herein regarding FIGS. 1 and 2A-2D.

According to a further embodiment, it may be possible to divide overweight people with abnormal blood sugar to a plurality of clinical groups based on, e.g., body mass index (BMI), age, weight, sex, blood pressure and other clinical characteristics as well as severity of abnormal conditions (e.g., how “abnormal” is a blood sugar disorder). Then each such a clinical group of the plurality of the clinical groups can have its own and more accurately determined threshold level for a number of daily steps, which can be determined using a procedure illustrated and discussed in reference to FIGS. 1 and 2A-2B. Then a subject/person can be matched to one of the clinical groups using known clinical characteristics of the person so that a corresponding more accurately determined threshold number of daily steps can be used for the physical activity of the person.

Furthermore, it follows from the above discussion that for low intensity physical activity (e.g., walking slower than 5 km/hour) with an acceleration range below of 1.1 g and possibly more precisely below of approximately 1.0 g and can be used for reducing a risk of disorders of sugar, blood cholesterol and fat metabolism of an overweight person with an abnormal blood sugar. In the study presented in FIGS. 1 and 2A-2D, the acceleration ranges below 0.3 g were not registered by the accelerometer. It is known in the art that a very slow walking (below 1 km/hour or a step about every 3-4 seconds) induces accelerations below 0.3 g, which were not registered, but still can make a difference. This may be considered in future studies and protocols. Thus, the lower level value can below 0.3 g (e.g., 0.2 g or below) or higher than 0.3 g (e.g., 0.4 g or even higher).

Based on the above considerations, various embodiments of the present invention are further disclosed below.

According to an embodiment of the invention, an apparatus, carried by a person during day activities (the person being overweight and having an abnormal blood sugar), comprising: an accelerometer (can be a uniaxial accelerometer or a polyaxial accelerometer) that continuously registers acceleration maximum values induced by a physical activity of the person; a processor; and a memory for storing the registered acceleration maximum values and tangibly storing computer program instructions capable of being executed by the processor. As an example, the overweight person can be obese with a body mass index (MBI) of 30 to 33 kg m⁻².

The computer program instructions can define code for determining selected acceleration maximum values out of the stored registered acceleration maximum values which are within at least one preset acceleration range corresponding to a low intensity physical activity comprising slow walking of said overweight person with a speed below 5 km/hour, where the at least one preset acceleration range being below 1.1 g and having a lower limit value and a high limit value (for example, the at least one preset acceleration range can be 0.3 to 0.7 g corresponding to walking speed of approximately 2-3 km/hour).

Moreover, the computer program instructions can further define code for calculating a number of daily steps of the person based on the selected acceleration maximum values, where one step corresponds to one selected acceleration maximum value, and comparing a total number of calculated steps for a predetermined period of time comprising one or more days or an average number of steps per day with a predefined threshold level (e.g., the predefined threshold level for the total number of steps in the at least one preset acceleration range of 0.3-0.7 g per day may be 6520 steps which is stored in the memory), and then provide a recommendation/results for the person to a display of the apparatus or to a wireless device associated with the person and communicating with the apparatus for presenting the recommendation on a screen of the wireless device, based on the above steps of determining and comparing, about maintaining or modifying the low intensity physical activity of the person including at least a required number of steps per day corresponding to slow walking of the person with the speed below 5 km/hour, in order to reduce a risk of disorders of sugar, blood cholesterol and fat metabolism of the person. In other words, if the number of selected acceleration maximum values has exceeded the predefined threshold level, the low intensity physical activity may prevent at least in part said disorders in sugar, cholesterol and fat metabolism.

According to a further embodiment, the wireless device may be a personal laptop computer, tablet, smartphone and the like and not carried by the person during the day. On the other hand, the apparatus may comprise a display which can be configured to provide a presentation of recommendation/results about maintaining or modifying the low intensity physical activity of the person, the recommendation results can be generated using the computer program instructions stored in the memory.

According to various embodiments as indicated herein, the lower limit value of the acceleration range can be predefined as approximately 0.3 g (but also higher or lower, e.g., 0.2 g or 0.4 g and the like) and the high limit value can be a predefined value from 0.5 g to 1.0 g. Furthermore, the at least one preset acceleration range can be chosen to be 0.3-0.9 g corresponding to the low intensity physical activity comprising slow walking of the overweight person with a speed below 5 km/hour being approximately 2 to 4 km/hour, so that the predefined threshold level for the total number of steps per day then is 6790.

According to another embodiment, the at least one preset acceleration range may be chosen based on matching clinical characteristics of the person to corresponding clinical characteristics of a plurality of clinical groups to select a corresponding clinical group, for each clinical group at least one corresponding predefined threshold level being stored in the memory, so that the stored at least one corresponding predefined threshold level becomes the predefined threshold level.

According to another embodiment, the at least one preset acceleration range and a corresponding predefined threshold level may be determined by a preliminary study of the physical activity of the person using the computer program instructions defining further code for determining preliminary selected acceleration maximum values of the person out of the stored registered acceleration maximum values which are within a plurality of acceleration ranges being below the acceleration value of 1.1 g comprising slow walking of said overweight person with the speed below 5 km/hour.

Moreover, the computer program instructions can further define code for calculating a preliminary number of daily steps of the person in each of the plurality of the acceleration ranges based on the preliminary selected acceleration maximum values, where one preliminary step corresponds to one preliminary selected acceleration maximum value, thus providing a distribution of the preliminary number of steps in the plurality of acceleration ranges below the acceleration value of 1.1 g; and then matching the provided distribution of the preliminary number of steps to a corresponding distribution out of a plurality corresponding distributions stored in the memory for a plurality clinical groups for each of which at least one corresponding predefined threshold level being further stored in the memory, and selecting a corresponding clinical group based on the matching, so that for the selected corresponding clinical group, the at least one corresponding predefined threshold becomes the predefined threshold level.

According to a further embodiment, further matching can be provided based on matching clinical characteristics of the person to corresponding clinical characteristics of the corresponding clinical group out of the plurality of clinical groups, so that both, the provided distribution of the preliminary number of steps and the clinical characteristics of the person, can be used for selecting the corresponding clinical group.

According to an embodiment of the invention, the apparatus may further comprise a wireless or wired transceiver for one or more of: 1) sending at least the stored registered acceleration maximum values to an outside computing device for a further analysis, and 2) receiving medical test results related to the sugar, blood cholesterol and fat metabolism of the person to perform the further analysis, wherein the further analysis comprises further comparing said total number of steps in the at least one preset acceleration range being below 1.1 g with the medical test results for providing a further recommendation to the person about maintaining or modifying his/her physical activities.

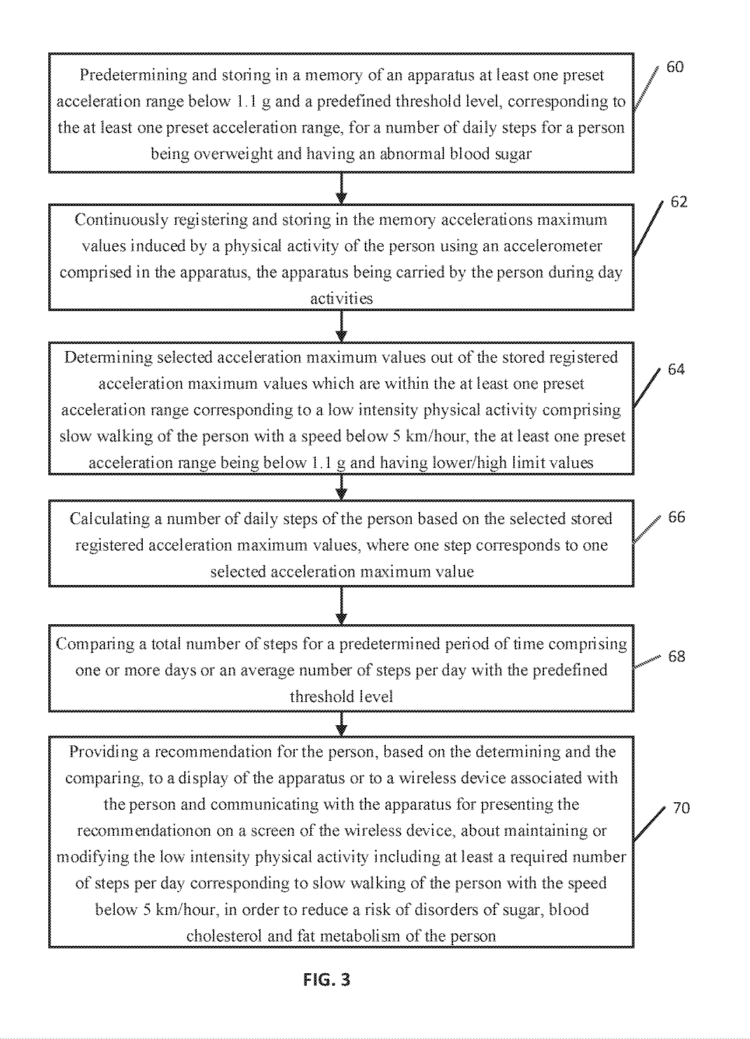

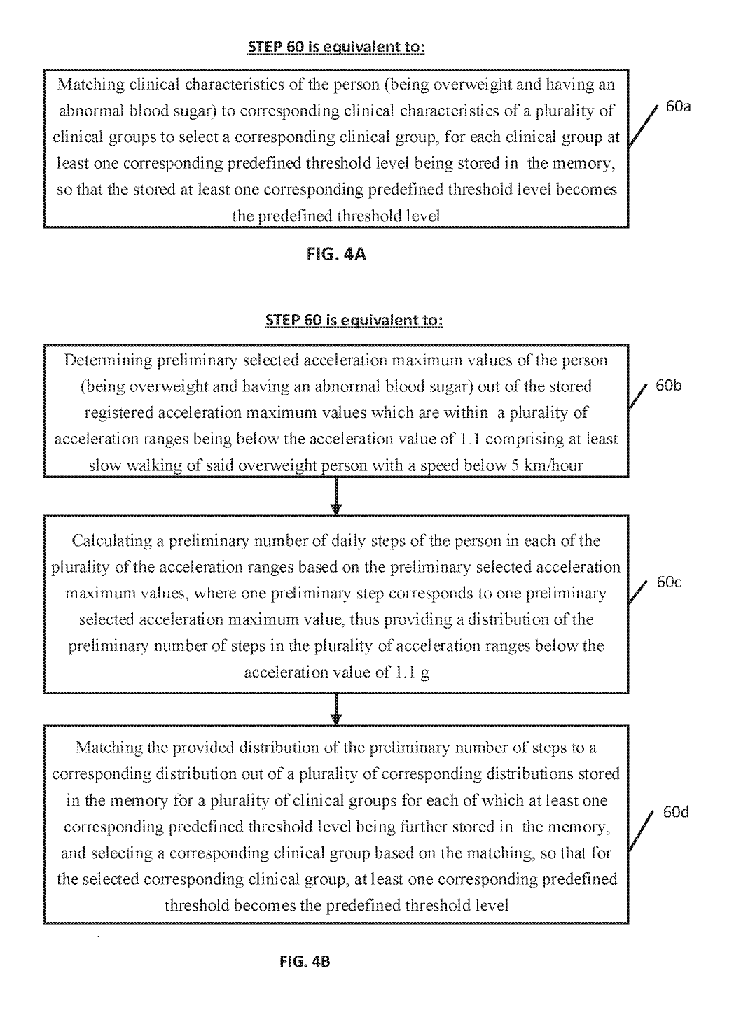

FIGS. 3 and 4A-4B are exemplary flowchart for demonstrating implementation of embodiments, disclosed herein. It is noted that the order of steps shown in FIGS. 3 and 4B is not required, so in principle, the various steps may be performed out of the illustrated order. Also, certain steps may be skipped, different steps may be added or substituted, or selected steps or groups of steps may be performed in a separate application, following the embodiments described herein.

In a method according to the exemplary embodiment, as shown in FIG. 3, in a first step 60, it is performed predetermining and storing in a memory of an apparatus 2 at least one (it could more than one) preset acceleration range below 1.1 g and a predefined threshold level (it could more than one), corresponding to the at least one preset acceleration range, for a number of daily steps for a person being overweight and having an abnormal blood sugar. In a next step 62, the apparatus is continuously registering and storing in the memory of the apparatus 2 accelerations maximum values induced by a physical activity of the person using an accelerometer 21 comprised in the apparatus, the apparatus being carried by the person during day activities.

In a next step 64, the apparatus is determining selected acceleration maximum values out of the stored registered acceleration maximum values which are within the at least one preset acceleration range corresponding to a low intensity physical activity comprising slow walking of the person with a speed below 5 km/hour, the at least one preset acceleration range being below 1.1 g and having lower/high limit values. In a next step 66, the apparatus is calculating a number of daily steps of the person based on the selected stored registered acceleration maximum values, where one step corresponds to one selected acceleration maximum value. Then in a step 68, the apparatus is comparing a total number of steps for a predetermined period of time comprising one or more days or an average number of steps per day with the predefined threshold level.

Finally in a step 70, the apparatus is providing recommendation/results for the person, based on the determining and the comparing, to a display of the apparatus or to a wireless device associated with the person and communicating with the apparatus for presenting the recommendation/results on a screen of the wireless device, about maintaining or modifying the low intensity physical activity including at least a required number of steps per day corresponding to slow walking of the person with the speed below 5 km/hour, in order to reduce a risk of disorders of sugar, blood cholesterol and fat metabolism of the person.

FIGS. 4A and 4B provide further embodiments/clarifications for performing step 60 in FIG. 3. FIG. 4A addresses a situation when it could be a plurality of clinical groups to select from. Then in step 60a (performed instead of step 60 in FIG. 1, the apparatus is matching clinical characteristics of the person (being overweight and having an abnormal blood sugar) to corresponding clinical characteristics of the plurality of clinical groups to select a corresponding clinical group, for each clinical group at least one corresponding predefined threshold level being stored in the memory, so that the stored at least one corresponding predefined threshold level for the matched clinical group becomes the predefined threshold level. It is further noted that in step 60 in FIG. 3, clinical characteristics of the person can be evaluated as well, to determine whether the person matches a clinical profile of a person being overweight and having an abnormal blood sugar.

FIG. 4B is a flowchart addressing a situation when it could be a plurality of clinical groups to select from and the selection is made based on an additional preliminary study for matching distribution of the preliminary number of steps in the plurality of acceleration ranges below the acceleration value of 1.1 g for the person (similar to the distribution shown in FIG. 1. In a step 60b, the apparatus is determining preliminary selected acceleration maximum values of the person (being overweight and having an abnormal blood sugar) out of the stored registered acceleration maximum values which are within a plurality of acceleration ranges identified below the acceleration value of 1.1 comprising slow walking of said overweight person with a speed below 5 km/hour.

In a next step 60c, the apparatus is calculating a preliminary number of daily steps of the person in each of the plurality of the acceleration ranges based on the preliminary selected acceleration maximum values, where one preliminary step corresponds to one preliminary selected acceleration maximum value, thus providing a distribution of the preliminary number of steps in the plurality of acceleration ranges below the acceleration value of 1.1 g.

In a next step 60d, the apparatus is matching the provided distribution of the preliminary number of steps to a corresponding distribution out of a plurality of corresponding distributions stored in the memory for a plurality of clinical groups for each of which at least one corresponding predefined threshold level being further stored in the memory, and selecting a corresponding clinical group based on the matching, so that for the selected corresponding clinical group, at least one corresponding predefined threshold becomes the predefined threshold level.

It is further noted that, the matching can be further provided based on matching clinical characteristics of the person to corresponding clinical characteristics of a clinical group out of the plurality of clinical groups, so that both, the provided distribution of the preliminary number of steps and the clinical characteristics of the person, may be used for selecting the corresponding clinical group.

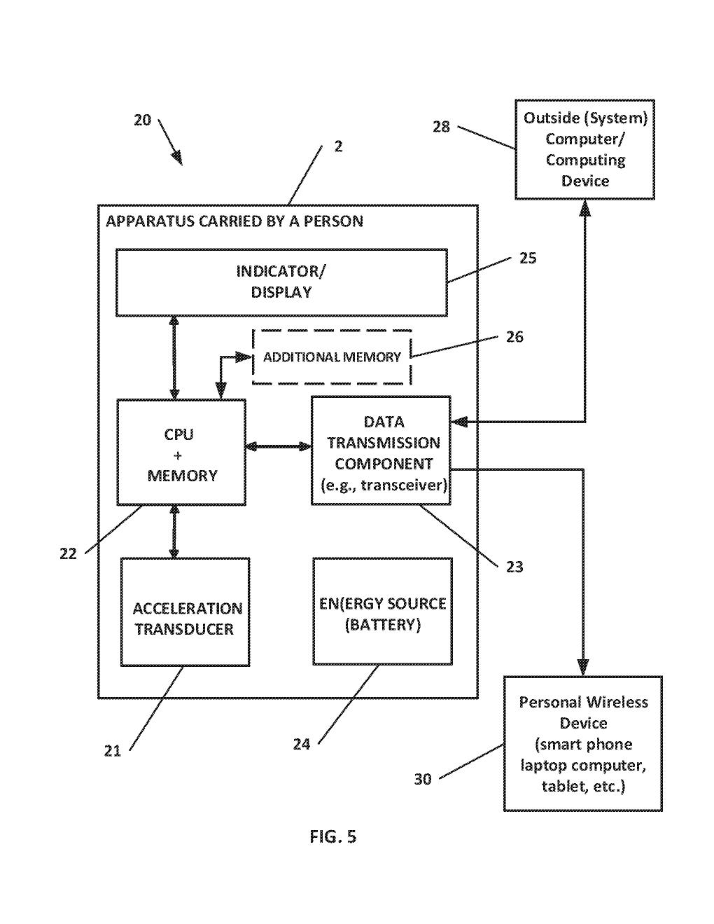

FIG. 5 is a block-diagram of a system 20 for implementing embodiments of the present invention described herein. FIG. 5 is a simplified block diagram that is suitable for practicing the exemplary embodiments of the present invention, and a specific manner in which components operate. The main device in the system 20 is an apparatus (transducer unit) 2 carried by a person/subject during day activities.

The transducer unit (apparatus) 2 advantageously comprises an energy source 24 such as an electric battery, accumulator, a rechargeable battery and the like. The electric elements contained by the transducer unit 2 get the energy required for their operation from this energy source 24. There is at least one acceleration transducer 21 (uniaxial or polyaxial accelerometer) in the transducer unit 2. By using more than one transducer 21, accelerations can be measured in two or three dimensions, when required. The measurement range of a single acceleration transducer is about AO g.

The measurement data of the acceleration transducer 21 are transmitted to a central processing unit (CPU) 22 of the transducer unit 2, which also advantageously comprises a certain amount of memory used by the processing unit 22 for saving various computer program applications/instructions and the results of performed acceleration measurements according to various embodiments of the invention. An additional memory 26, if needed, can be also used for the same purposes. The processing unit 22 is configured to execute the stored computer program instructions to implement steps of flow charts presented in FIGS. 3 and 4A-4B including an analysis of the measured acceleration data.

The processing unit 22 can also communicate with a data transfer/transmission component 23 (e.g., a wireless transceiver). Using this data transfer component 23, a data transfer connection can be set up to the external (system) data processing computer/computing device 28 to send recorded data and to receive corresponding medical test results. The data transfer/transmission component 23 can supports various communication methods used in data transmission including infrared (IR) technique, Bluetooth technique, WLAN technique and various time or code division data transfer techniques used in cellular networks and the like.

Moreover, in another embodiment, the data transfer/transmission component 23 is configured to provided recommendation/results (generated by the processing unit 22) for the person, based on the determining and the comparing steps in FIG. 3, to a display 25 of the apparatus 2 or to a wireless device 30 associated with the person and communicating with the apparatus 2 for presenting the recommendation/results on a screen of the wireless device 30 (such as a personal laptop computer, smartphone, tablet and the like) about maintaining or modifying the low intensity physical activity including at least a required number of steps per day corresponding to slow walking of the person.

The transducer unit 2 can further comprise an indicator (display) 25, e.g. for indicating the result of the first analysis specific to the time of use. By means of the indicator, it is possible to communicate to the user of the transducer unit 2 e.g. simple messages of ON/OFF type if the exercise performed has promoted cholesterol metabolism. The indicator 25 can be e.g. LEDs of different colors. If the objective of the exercise performed is reached, a LED of certain color would light up. Alternatively, the indicator 25 can comprise exclusively a display (such as an LED display) to provided recommendations/results described herein (e.g., day-specific or week-specific information).

The memory (or at least one memory) can be an operational memory of the processor 22 or it can be a separate additional memory 26 or multiple additional memories, depending on implementation needs. Various embodiments for the memories (e.g., computer readable memory) may include any data storage technology type which is suitable to the local technical environment, including but not limited to: semiconductor based memory devices, magnetic memory devices and systems, optical memory devices and systems, fixed memory, removable memory, disc memory, flash memory, DRAM, SRAM, EEPROM and the like. Various embodiments of the processor 22 may include but are not limited to general purpose computers, special purpose computers, microprocessors, digital signal processors (DSPs) and multi-core processors.

The invention presented herein has many advantages. It makes it possible to determine for the first time the volume and intensity of the physical activity threshold reducing disease risks in subjects, who are sedentary, being overweight and suffering from metabolic disorders. These subjects cover approximately 50% of the population and previously there have not been any physical activity recommendations for them. The accelerometer described in the intervention can be easily used continuously making it possible to register all types of physical activity including habitual activities. This will make it easy to meet our novel physical activity recommendations. From the point of preventive medicine, walking is also the best form of physical activity. It activates the biggest muscles in the body and its energy expenditure can be accurately measured by accelerometers recording number of steps and step induced accelerations or walking speeds. Maintaining walking ability can also protect from falling accidents and hospitalization.

High occurrence of overweight, obesity and metabolic disorders in the present population and the information of the preventive characteristics and efficacy of the invention described here will offer a perspective for extensive use and for commercial adaptations.

The invention described herein is significantly different from the previously known methods by which exercises have been used for prevention of risk factors of diabetes and cardiovascular diseases. Physicians have generally been aware about the beneficial effects of regular exercise, but the volume, intensity and duration of the exercises have been unknown. It was novel and unexpected in our invention that for sedentary subjects, physical activity of very low intensity (e.g., with accelerations 0.3-0.7 g) can reduce concentrations of the major risk factors of diabetes and cardiovascular diseases. This invention is also different form other patents and recommendations, since those do not take low intensity exercise into account.

Unless defined otherwise, technical and scientific terms used herein have the same meaning as is commonly understood by one having ordinary skill in the art to which this disclosure belongs. The terms “first”, “second”, and the like, as used herein, do not denote any order, quantity, or importance, but rather are employed to distinguish one element from another. Also, the terms “a” and “an” do not denote a limitation of quantity, but rather denote the presence of at least one of the referenced items. The use of “including,” “comprising” or “having” and variations thereof herein are meant to encompass the items listed thereafter and equivalents thereof, as well as additional items. The terms “connected” and “coupled” are not restricted to physical or mechanical connections or couplings, and can include electrical and optical connections or couplings, whether direct or indirect.

Furthermore, the skilled artisan will recognize the interchangeability of various features from different embodiments. The various features described, as well as other known equivalents for each feature, can be mixed and matched by one of ordinary skill in this art, to construct additional systems and techniques in accordance with principles of this disclosure.

In describing alternate embodiments of the apparatus claimed, specific terminology is employed for the sake of clarity. The invention, however, is not intended to be limited to the specific terminology so selected. Thus, it is to be understood that each specific element includes all technical equivalents that operate in a similar manner to accomplish similar functions.

It is to be understood that the foregoing description is intended to illustrate and not to limit the scope of the invention, which is defined by the scope of the appended claims. Other embodiments are within the scope of the following claims.

It is noted that various non-limiting embodiments described and claimed herein may be used separately, combined or selectively combined for specific applications. Further, some of the various features of the above non-limiting embodiments may be used to advantage, without the corresponding use of other described features. The foregoing description should therefore be considered as merely illustrative of the principles, teachings and exemplary embodiments of this invention, and not in limitation thereof

1. An apparatus comprising:
an accelerometer that continuously registers accelerations maximum values induced by a physical activity of a person, said apparatus is carried by the person during day activities, the person being overweight and having an abnormal blood sugar;
a processor; and
a memory for storing the registered acceleration maximum values and tangibly storing computer program instructions capable of being executed by the processor, the computer program instructions defining code for:
determining selected acceleration maximum values out of the stored registered acceleration maximum values which are within at least one preset acceleration range corresponding to a low intensity physical activity comprising slow walking of said overweight person with a speed below 5 km/hour, the at least one preset acceleration range being below 1.1 g and having a lower limit value and a high limit value;
calculating a number of daily steps of the person based on the selected acceleration maximum values, where one step corresponds to one selected acceleration maximum value;
comparing a total number of steps for a predetermined period of time comprising one or more days or an average number of steps per day with a predefined threshold level; and
providing a recommendation for the person, based on the determining and the comparing, to a display of the apparatus or to a wireless device associated with the person and communicating with the apparatus for presenting the recommendation on a screen of the wireless device, about maintaining or modifying the low intensity physical activity of the person including at least a required number of steps per day corresponding to slow walking of the person with the speed below 5 km/hour, in order to reduce a risk of disorders of sugar, blood cholesterol and fat metabolism of the person.

2. The apparatus of claim 1, wherein the lower limit value is approximately 0.3 g and the high limit value is a predefined value from 0.5 g to 1.0 g.

3. The apparatus of claim 1, wherein the at least one preset acceleration range is 0.3-0.7 g corresponding to the low intensity physical activity comprising slow walking of the person with a speed of approximately 2 to 3 km/hour, wherein the predefined threshold level for the total number of steps per day in the acceleration range of 0.3-0.7 g is 6520.

4. The apparatus of claim 1, wherein the at least one preset acceleration range is 0.3-0.9 g corresponding to the low intensity physical activity comprising slow walking of said overweight person with a speed below 5 km/hour being approximately 2 to 4 km/hour, wherein the predefined threshold level for the total number of steps per day in the acceleration range of 0.3-0.9 g is 6790.

5. The apparatus of claim 1, wherein the at least one preset acceleration range is chosen based on matching clinical characteristics of the person to corresponding clinical characteristics of a plurality of clinical groups to select a corresponding clinical group, for each clinical group at least one corresponding predefined threshold level being stored in the memory, so that the stored at least one corresponding predefined threshold level becomes the predefined threshold level.

6. The apparatus of claim 1, wherein the at least one preset acceleration range and a corresponding predefined threshold level are determined by a preliminary study of the physical activity of the person using the computer program instructions defining further code for:
determining preliminary selected acceleration maximum values of the person out of the stored registered acceleration maximum values which are within a plurality of acceleration ranges being below the acceleration value of 1.1 g comprising slow walking of said overweight person with the speed below 5 km/hour;
calculating a preliminary number of daily steps of the person in each of the plurality of the acceleration ranges based on the preliminary selected acceleration maximum values, where one preliminary step corresponds to one preliminary selected acceleration maximum value, thus providing a distribution of the preliminary number of steps in the plurality of acceleration ranges below the acceleration value of 1.1 g; and
matching the provided distribution of the preliminary number of steps to a corresponding distribution out of a plurality of corresponding distributions stored in the memory for a plurality of clinical groups for each of which at least one corresponding predefined threshold level being further stored in the memory, and selecting a corresponding clinical group based on the matching, so that for the selected corresponding clinical group, the at least one corresponding predefined threshold becomes the predefined threshold level.

7. The apparatus of claim 6, wherein further matching is provided based on matching clinical characteristics of the person to corresponding clinical characteristics of the corresponding clinical group out of the plurality of clinical groups, so that both, the provided distribution of the preliminary number of steps and the clinical characteristics of the person, are used for selecting the corresponding clinical group.

8. The apparatus of claim 1, wherein, if the number of selected acceleration maximum values has exceeded the predefined threshold level, the low intensity physical activity has prevented at least partly said disorders in sugar, cholesterol and fat metabolism.

9. The apparatus of claim 1, further comprising a wireless or wired transceiver for one or more of: 1) sending at least the stored registered acceleration maximum values to an outside computing device for a further analysis, and 2) receiving medical test results related to the sugar, blood cholesterol and fat metabolism of the person to perform the further analysis, wherein the further analysis comprises further comparing said total number of steps in the at least one preset acceleration range being below 1.1 g with the medical test results for providing a further recommendation to the person about maintaining or modifying the physical activities of the person.

10. A method performed by an apparatus comprising:
continuously registering acceleration maximum values induced by a physical activity of a person using an accelerometer of the apparatus, where the apparatus is carried by the person during day activities, the person being overweight and having an abnormal blood sugar;
storing the registered acceleration maximum values in a memory of the apparatus;
determining selected acceleration maximum values out of the stored registered acceleration maximum values which are within at least one preset acceleration range corresponding to a low intensity physical activity comprising slow walking of said overweight person with a speed below 5 km/hour, the at least one preset acceleration range being below 1.1 g and having a lower limit value and a high limit value, as executed by a processor of the apparatus using computer program instructions stored in the memory;
calculating a number of daily steps of the person based on the selected acceleration maximum values, where one step corresponds to one selected acceleration maximum value, as executed by the processor of the apparatus using the computer program instructions stored in the memory;
comparing a total number of steps for a predetermined period of time comprising one or more days or an average number of steps per day with a predefined threshold level, as executed by the processor of the apparatus using the computer program instructions stored in the memory; and
providing a recommendation for the person, based on the determining and the comparing, to a display of the apparatus or to a wireless device associated with the person and communicating with the apparatus for presenting the recommendation on a screen of the wireless device, about maintaining or modifying the low intensity physical activity of the person including at least a required number of steps per day corresponding to slow walking of the person with the speed below 5 km/hour, in order to reduce a risk of disorders of sugar, blood cholesterol and fat metabolism of the person, as executed by the processor of the apparatus using the computer program instructions stored in the memory.

11. The method of claim 10, wherein the lower limit value is approximately 0.3 g and the high limit value is a predefined value from 0.5 g to 1.0 g.

12. The method of claim 10, wherein the at least one preset acceleration range is 0.3-0.7 g corresponding to the low intensity physical activity comprising slow walking of the person with a speed of approximately 2 to 3 km/hour, wherein the predefined threshold level for the total number of steps per day in the acceleration range of 0.3-0.7 g is 6520.

13. The method of claim 10 wherein the at least one preset acceleration range is 0.3-0.9 g corresponding to the low intensity physical activity comprising slow walking of said overweight person with a speed below 5 km/hour being approximately 2 to 4 km/hour, wherein the predefined threshold level for the total number of steps per day in the acceleration range of 0.3-0.9 g is 6790.

14. The method of claim 10, wherein the at least one preset acceleration range is chosen based on matching clinical characteristics of the person to corresponding clinical characteristics of a plurality of clinical groups to select a corresponding clinical group, for each clinical group at least one corresponding predefined threshold level being stored in the memory, so that the stored at least one corresponding predefined threshold level becomes the predefined threshold level.

15. The method of claim 10, wherein the at least one preset acceleration range and a corresponding predefined threshold level are determined by a preliminary study of the physical activity of the person using the computer program instructions defining further code for:
determining preliminary selected acceleration maximum values of the person out of the stored registered acceleration maximum values which are within a plurality of acceleration ranges being below the acceleration value of 1.1 g comprising slow walking of said overweight person with the speed below 5 km/hour;
calculating a preliminary number of daily steps of the person in each of the plurality of the acceleration ranges based on the preliminary selected acceleration maximum values, where one preliminary step corresponds to one preliminary selected acceleration maximum value, thus providing a distribution of the preliminary number of steps in the plurality of acceleration ranges below the acceleration value of 1.1 g; and
matching the provided distribution of the preliminary number of steps to a corresponding distribution out of a plurality of corresponding distributions stored in the memory for a plurality of clinical groups for each of which at least one corresponding predefined threshold level being further stored in the memory, and selecting a corresponding clinical group based on the matching, so that for the selected corresponding clinical group, the at least one corresponding predefined threshold becomes the predefined threshold level.

16. The method of claim 15, wherein further matching is provided based on matching clinical characteristics of the person to corresponding clinical characteristics of the corresponding clinical group out of the plurality of clinical groups, so that both, the provided distribution of the preliminary number of steps and the clinical characteristics of the person, are used for selecting the corresponding clinical group.

17. The method of claim 14, wherein, if the number of selected acceleration maximum values has exceeded the predefined threshold level, the low intensity physical activity has prevented at least partly said disorders in sugar, cholesterol and fat metabolism.

18. The method of claim 11, further comprising:
sending, by a wireless or wired transceiver of the apparatus, at least the stored registered acceleration maximum values to an outside computing device for a further analysis using medical test results, related to the sugar, blood cholesterol and fat metabolism of the person to generate further recommendations, and for providing the further recommendations to the person about maintaining or modifying the physical activities of the person.

19. The method of claim 11, further comprising:
receiving, by a wireless or wired transceiver of the apparatus, medical test results related to said sugar, blood cholesterol and fat metabolism of the person to perform a further analysis and to generate further recommendations, and for providing the further recommendations to the person about maintaining or modifying the physical activities of said overweight person.

20. A non-transitory computer readable storage medium tangibly storing computer program instructions capable of being executed by a computer processor, the computer program instructions defining code for:
continuously registering acceleration maximum values induced by a physical activity of a person using an accelerometer of the apparatus, where the apparatus is carried by the person during day activities, the person being overweight and having an abnormal blood sugar;
storing the registered acceleration maximum values in a memory of the apparatus;
determining selected acceleration maximum values out of the stored registered acceleration maximum values which are within at least one preset acceleration range corresponding to a low intensity physical activity comprising slow walking of said overweight person with a speed below 5 km/hour, the at least one preset acceleration range being below 1.1 g and having a lower limit value and a high limit value;
calculating a number of daily steps of the person based on the selected acceleration maximum values, where one step corresponds to one selected acceleration maximum value;
comparing a total number of steps for a predetermined period of time comprising one or more days or an average number of steps per day with a predefined threshold level; and
providing a recommendation for the person, based on the determining and the comparing, to a display of the apparatus or to a wireless device associated with the person and communicating with the apparatus for presenting the recommendation on a screen of the wireless device, about maintaining or modifying the low intensity physical activity of the person including at least a required number of steps per day corresponding to slow walking of the person with the speed below 5 km/hour, in order to reduce a risk of disorders of sugar, blood cholesterol and fat metabolism of the person, as executed by the processor of the apparatus using the computer program instructions stored in the memory.
